(19) United States
(12) Patent Application Publication
(10) Pub. No.: US 2022/0047495 A1
(43) Pub. Date: Feb. 17, 2022

(54) ROSEWOOD EXTRACT

(71) Applicant: L V M H Recherche, Saint Jean de Braye (FR)

(72) Inventors: Jocelyne FRANCHI, SAINT JEAN DE LA RUELLE (FR); Virginie PECHER, LA CHAPELLE SAINT MESMIN (FR); Milène JUAN, SAINT-JEAN DE BRAYE (FR)

(73) Assignee: L V M H Recherche, Saint Jean de Braye (FR)

(21) Appl. No.: 17/415,698

(22) PCT Filed: Dec. 10, 2019
(86) PCT No.: PCT/EP2019/084365

§ 371 Date: Jun. 17, 2021

(30) Foreign Application Priority Data
Dec. 20, 2018 (FR) FR1873706

(51) Int. Cl. A61K 8/9789; A61Q 19/00; A61Q 19/08

(52) U.S. Cl. CPC A61K 8/9789; A61Q 19/007; A61K 2800/805; A61K 2800/522; A61Q 19/08

(57) ABSTRACT

The invention relates to an aqueous extract of rosewood, preferably rosewood from summer roses, obtained by enzymatic extraction using a cosmetically acceptable polar solvent, a process for producing same, and uses thereof, particularly as an agent for preventing and/or reducing the signs of aging on the skin.

### FIELD OF THE INVENTION

The present invention relates to an aqueous extract of rosewood, in particular of the Evanrat or Jardin de Granville® rose variety, and to uses thereof as an agent intended to prevent and/or treat the signs of skin aging, in particular of the skin of the face and/or body.

### STATE OF THE ART

The skin is the body's initial protective barrier against the environment. It is therefore subject to factors of exogenous origin (e.g., UV radiation, temperature variations, atmospheric pollution, cigarette smoke, etc.) or endogenous origin (e.g., hormones, etc.) that are likely to induce signs of skin aging, which are referred to as extrinsic aging or intrinsic (or physiological) aging, respectively.

Extrinsic aging leads to clinical alterations such as deep wrinkles and the formation of skin that has lost its firmness, suppleness and elasticity, the alteration of the barrier function of the skin, the alteration of the innate immunity of the skin and the appearance of cutaneous pigment spots, also called age spots, brown spots, solar lentigo, actinic lentigo or senile lentigo, particularly on the back of the hands, the face, the neckline or, in men, the skull. Dry skin is also known to be lackluster and more sensitive to pigmentary disorders. At the same time, intrinsic or physiological aging causes a slowing down in the renewal of skin cells, the keratinocytes, which is essentially expressed as the appearance of clinical alterations such as the reduction of subcutaneous adipose tissue and the appearance of fine lines or wrinkles.

Endogenous and/or exogenous stresses can also be a major cause of inflammaging (or inflamm-aging), which is a contraction of the terms “inflammation” and “aging”. This chronic and silent process of inflammation, deciphered by Franceschi C et al. in 2000 (Inflamm-aging. An evolutionary perspective on immunosenescence. Annals of the New York Academy of Sciences. 2000; 908:244-254), is described as a natural defense reaction of the immune system to stress that degenerates into a chronic and silent microinflammatory process, responsible notably for the aging of keratinous materials and in particular the skin. With age, repetitive microinflammations spread in the skin tissue and weaken the cells' defense systems, resulting in the appearance of wrinkles, sagging skin, dry skin, and/or a dull and uneven complexion. This phenomenon increases with time and affects all skin types, particularly sensitive skin, which is more fragile due to stress and external insults.

With age, the epidermis, which is thinner and more fragile, is also more permeable to these insults, which generate stress in its cells and unbalance its natural state of homeostasis. The skin's immune system defends itself and triggers the release, inter alia and in excess, of free radicals, neuropeptides and proinflammatory mediators (cytokines), which lead to the degradation of collagen and elastin fibers, slow down the capacity for cell renewal, and accelerate skin aging, thus leading to a loss of tone, elasticity and firmness with an alteration of the barrier function.

There is therefore a search for new anti-aging ingredients capable of preventing and/or reducing the above-mentioned signs of skin aging.

Unexpectedly, the Applicant has shown the in vitro effects of an aqueous extract of rosewood, in particular of Jardin de Granville® rose, making it possible to envisage its use as an active agent to prevent and/or treat skin aging, in particular induced by oxidative stress, to prevent and/or reduce an alteration of the barrier function of the skin, to prevent and/or reduce the appearance of cutaneous pigmentation spots, to prevent and/or reduce an alteration in the innate immunity of the skin or to prevent and/or reduce the signs of skin aging linked to chronic microinflammation of the skin, also known as ‘inflammaging’.

### SUMMARY OF THE INVENTION

A first subject matter of the invention relates to an aqueous extract of rosewood, preferably of summer rosewood, obtained by enzymatic extraction using a cosmetically acceptable polar solvent.

Another subject matter of the invention relates to a process for preparing a rosewood extract in accordance with the invention comprising the steps:
a) addition of water to plant material with a suitable particle size,
b) addition of an enzyme mixture containing at least one cellulase, at least one hemicellulase and at least one pectinase, and advantageously also at least one protease,
c) incubation under stirring of the plant material and the enzyme mixture to release in the reaction medium oils, proteins and fermentable sugars, for a time depending on the desired yields,
d) separation of the reaction medium to obtain free oil, an aqueous phase containing proteins and fermentable sugars, and a solid phase, and
e) separation of the aqueous phase and optional addition of preservatives.

The invention also relates to a cosmetic composition for topical application to the skin and/or lips, in particular the skin of the face and/or body, comprising, in a physiologically acceptable medium, at least an effective amount of at least one aqueous extract of rosewood in accordance with the invention.

Another subject matter of the invention is the non-therapeutic cosmetic use of at least one aqueous extract of rosewood in accordance with the invention, in particular of the Evanrat or Jardin de Granville® rose variety, as an agent intended to prevent and/or reduce the signs of skin aging, in particular the loss of tone, the loss of elasticity and/or the loss of firmness of the skin, the alteration of the barrier function, the appearance of wrinkles and/or fine lines, the appearance of cutaneous pigment spots, in particular senile lentigo (age spots) or solar lentigo, dry skin, and/or a dull and uneven complexion.

It also relates to a non-therapeutic cosmetic process intended to prevent and/or reduce the signs of skin aging, in particular the loss of tone, the loss of elasticity and/or the loss of firmness of the skin, the alteration of the barrier function, the appearance of wrinkles and/or fine lines, the appearance of cutaneous pigment spots, in particular senile lentigo (age spots) or solar lentigo, dry skin, and/or a dull and uneven complexion, characterized in that it comprises the application to the skin of the face and/or body of a cosmetic composition comprising an aqueous extract of rosewood as defined in the invention.

The expression “dull and uneven complexion” means in particular a complexion that lacks light and radiance, an altered, blurred complexion, presenting surface and/or color irregularities, generally associated with a perception of discomfort and manifestations felt as skin imperfections or aesthetic disorders.

Another subject matter of the invention is an aqueous extract of rosewood in accordance with the invention or a composition comprising same in accordance with the invention for use in soothing the skin and/or preventing and/or treating skin disorders related to chronic microinflammation of the skin, in particular preventing and/or reducing signs of skin aging induced by chronic microinflammation of the skin.

The expression ‘chronic microinflammation of the skin’ means invisible (silent) microinflammatory processes that are set up locally in the skin and which, if they degenerate or are poorly resolved, become chronic. Chronic microinflammation of the skin can in particular be related to the accumulation of tissue damage, immune system failure, or the accumulation of senescent and proinflammatory cytokine secreting cells. Over time, these repeated microinflammatory events contribute to skin aging.

### DETAILED DESCRIPTION OF THE INVENTION

Rosewood Extract and Preparation Process

A first subject matter of the invention relates to an aqueous extract of rosewood, preferably summer rosewood, obtained by enzymatic extraction using a cosmetically acceptable polar solvent.

The terms aqueous extract and polar extract and hydrophilic rose extract and eco-extract will be used interchangeably in the description. The aqueous extract of rosewood is advantageously obtained by means of a cosmetically acceptable polar solvent.

The term “rosewood extract” or “aqueous extract of rosewood” in accordance with the invention is understood to mean in particular that the polar (hydrophilic) compounds of the rosewoods have been solubilized and/or extracted in a polar solvent. Advantageously, this rosewood extract is obtained by an eco-extraction process. This is therefore referred to as rosewood eco-extract.

The expression “polar solvent” means that the solvent has a polarity index value of 4 or higher. The polarity index is a quantity calculated on the basis of thermodynamic quantities (of solubility and change of state) which shows the relatively polar nature of a molecule. For solvent polarity indices, see the paper by L.R. SNYDER: Classification of the solvent properties of common liquids; Journal of Chromatography, 92 (1974), 223-230.

Preferred polar solvents are those consisting of a compound comprising at least one O-H type polar covalent bond.

As cosmetically acceptable polar solvent, a solvent or a mixture of solvents chosen from water, C₁-C₄ alcohols such as ethanol, glycols such as ethylene glycol, glycerol, butylene glycol and propylene glycol, and mixtures thereof, is chosen. Preferably, water is used.

According to a particular embodiment, said extract is characterized in that it comprises water in a content ranging from 94% to 96%, a dry extract content ranging from 3% to 5% and preservatives in a content ranging from 0.5% to 1% by weight based on the total weight of the extract.

Plant Material

The extracts of the invention are rosewood extracts. In accordance with the invention, a rose extract, a rosewood extract or an extract in particular of the Evanrat or Jardin de Granville® rose variety will be referred to interchangeably.

The person skilled in the art will preferably choose selected roses whose properties are preserved by an environment and an organic farming method. According to a particular and preferred embodiment, an extract of rose petals of the Evanrat or ‘Jardin de Granville®’ rose variety will be used.

The ‘Jardin de Granville®’ rose is a hybrid variety offered exclusively by “Roses anciennes André Eve SAS” and protected by Certificate of Plant Breeders' Rights under no. 20110345 with for species the denomination Rosa L. and for variety the denomination EVANRAT. This rosebush belongs to the group of modern hybrids, which, from May to October, is permanently covered with roses, thus showing an excellent remontant character.

Thus, according to a particular and preferred embodiment of the invention, the aqueous extract of rosewood, also called rosewood extract, is an extract of rosewood of the Evanrat or Rose Jardin de Granville® variety.

There are two types of rosewood, based on the time of the year they are harvested:
Winter wood, generally harvested between November and April
Summer wood, generally harvested between May and October.

According to a particular and preferred embodiment, summer wood will be used. Rosewood or summer rosewood will be referred to interchangeably in the description.

The rosewoods in accordance with the invention are obtained from freshly cut whole roses and is isolated from the rest of the plant by manual or mechanical separation.

In a particular embodiment, the rosewoods are used directly for extraction.

In another embodiment, the rosewoods are pre-dried before extraction. According to a particular and preferred embodiment, the rosewood is ground prior to the extraction step.

Rosewoods contain monosaccharide sugars (fructose, glucose) and polysaccharides, especially disaccharides, polyphenols (catechin), amino acids (mostly aspartic acid, tyrosine and arginine), minerals (ash, sodium, potassium, calcium), flavonoids (mostly glycosylated) and tannins.

In particular, calcium improves epidermal differentiation and strengthens the skin structure, while potassium amplifies skin hydration and boosts energy assimilation. Plant sugars (fructose, glucose) are intended to fill the skin cells with energy.

The aqueous extract of rosewood in accordance with the invention is an extract concentrated in natural polar compounds. The aqueous extract of rosewood in accordance with the invention is distinct from a rosewater. Here, it is a rosewood extract involving the extraction of natural rosewood compounds in the presence of aqueous (polar) solvents with or without the addition of a pH modifier, and implementation, in the formulation, in the form of an aqueous solution or concentrate, it being possible for the solvent of the concentrate to be the extraction solvent and/or an additional solvent.

According to a particular embodiment, the aqueous extract of rosewood in accordance with the invention further comprises in a small proportion a rose flower extract representing from 0.01% to 1%, preferably from 0.05% to 0.5%, preferably from 0.1% to 0.2% of the total aqueous extract of rose in accordance with the invention. In this embodiment, the original plant material comprises rosewood and rose flowers in a proportion from 0.01% flowers to 99.99% wood to 1% flowers to 99% wood.

Extraction Process

The aqueous extract of rosewood in accordance with the invention can be obtained according to various eco-extraction processes known to the skilled person and in particular that described below.

According to a particular and preferred embodiment, rosewoods of the Evanrat variety, preferably the Jardin de Granville® rose, preferably pre-dried rosewoods, will be used as plant material in the extraction processes adapted to the invention described below.

“Eco-extraction” is understood to mean an extraction process that reduces energy consumption but also allows the use of alternative solvents and renewable plant resources, while ensuring a safe and quality product/extract. Eco-extraction promotes six main principles (Farid Chemat, Dunod, 2010, Eco-extraction du vegetal):
Promote innovation through varietal selection and the use of renewable plant resources,
Favor alternative, so-called green solvents and mainly those from agro-resources,
Reduce energy consumption through the use of innovative technologies and promote energy recovery,
Promote the creation of co-products instead of waste to integrate the organic or agro-refinery stream,
Reduce unit operations through technological innovation and promote safe, robust and controlled processes, and
Favor a product that is non-denatured, biodegradable, contaminant-free and, above all, that has “extract” values. Water will therefore advantageously be used as green polar solvent.

“Solid/liquid extraction” is understood to mean a type of extraction involving contact between a solid (bio-based material) and a liquid (called solvent) in order to allow one or more soluble compound(s) called solute(s) to diffuse/impregnate in the liquid. This is generally done through relatively rapid physical and physicochemical actions.

The woods can be used fresh, frozen or pre-dried using conventional drying tools known to the skilled person, such as air drying, oven drying, freeze drying or zeolite drying. Thus, according to an embodiment, and prior to the extraction step itself, the woods may have been dried and/or ground. According to another embodiment, the woods are used wet and then ground.

According to a particular embodiment, the wood is ground before extraction, for example by means of a mortar, a cryo-grinder, a blender, a conventional grinder or a centrifuge according to the methods known to the person skilled in the art.

The aqueous extract of rosewood in accordance with the invention can be obtained in particular by enzymatic extraction processes.

The selected enzymes hydrolyze some or all of the compounds in the plant material in order to damage the cell wall and thus favorably release the contained molecules.

“Cell wall” or “plant wall” or “pectocellulosic wall” is understood to mean the cellular structural element that protects each plant cell. It is composed of three parts in particular:
the middle lamella: rich in pectin and devoid of cellulose,
the primary wall: composed predominantly of cellulose (9% to 25%), hemicellulose (25% to 58%), pectins (10% to 35%), and proteins (10%),
the secondary wall: cellulose, hemicellulose.

Depending on the molecules to be extracted, the enzymes will be adapted to the walls to be hydrolyzed in order to access these molecules. Advantageously, enzymes of the cellulase, hemicellulase, pectinase or protease type can be used, alone or in combination.

“Cellulase” is understood to mean an enzyme that can break down cellulose, a biopolymer consisting of a linear chain of D-glucose and the main component of the plant cell wall. The end product of cellulose degradation by cellulase is glucose. There are several types of cellulases, such as beta-glucosidase, endocellulase, exocellulase, oxidative cellulase or cellulose phosphorylase.

“Hemicellulase” is understood to mean an enzyme that can break down hemicellulose, a biopolymer that is a constituent of the plant cell wall, allowing in particular bridging between cellulose fibers, as well as with other matrix compounds. There are several types of hemicellulases, such as arabinase, xylanase, or galactanase.

“Pectinase” is understood to mean an enzyme capable of degrading pectin, a polysaccharide present in plant cell walls. There are several types of pectinases, such as endopolygalacturonase, or pectin methylesterase.

“Protease” is understood to mean an enzyme capable of breaking down peptide bonds. Proteases are used in the process of the invention to hydrolyze structural proteins and improve lysis of the raw material, not to produce peptides.

Thus, a cellulase can be used to release glucose and cellulose; a hemicellulase to release mono- and oligomers of reduced sugars; a pectinase to release uronic acids; and advantageously a protease to hydrolyze structural proteins and improve the lysis of the raw material.

In a preferred embodiment, eco-extraction is performed using the protocol described in patent application WO2011045387 (corresponding to patent application FR2351461) on behalf of the Institut National Polytechnique de Lorraine INPL. It is an alternative technology that meets regulatory and environmental constraints while remaining economically competitive. This innovative process makes it possible to extract three types of products from plant matter in a single step, namely an oil (rich in nutrients such as polyphenols, sterols, vitamin E, etc.), an aqueous extract and a cake (solid phase).

This process comprises in particular the following steps:
a) addition of water to plant material with a suitable particle size,
b) addition of an enzyme mixture containing at least one cellulase, at least one hemicellulase and at least one pectinase, and advantageously also at least one protease,
c) incubation under stirring of the plant material and the enzyme mixture to release in the reaction medium oils, proteins and fermentable sugars, for a time depending on the desired yields,
d) separation of the reaction medium to obtain free oil, an aqueous phase containing proteins and fermentable sugars, and a solid phase, and
e) separation of the aqueous phase and optional addition of preservatives.

Thus, an aqueous phase corresponding to the rosewood extract in accordance with the invention is obtained at the end of the separation step (e).

Thus, another subject matter of the invention relates to a process for preparing a rosewood extract in accordance with the invention comprising the steps:
a) addition of water to the pre-ground rosewood with a suitable particle size,
b) addition of an enzyme mixture containing at least one cellulase, at least one hemicellulase and at least one pectinase, and advantageously also at least one protease,
c) incubation under stirring of the pre-ground rosewood and the enzyme mixture to release in the reaction medium oils, proteins and fermentable sugars, for a time depending on the desired yields,
d) separation of the reaction medium to obtain free oil, an aqueous phase containing proteins and fermentable sugars, and a solid phase, and
e) separation of the aqueous phase and optional addition of preservatives.

Said rosewood extract in accordance with the invention being obtained after the separation step (e).

Advantageously, this extraction process does not involve organic solvents, such as hexane, which could lead to a number of problems related to safety of facilities and personnel, human health and preservation of the environment. This technology reduce the emissions of volatile organic compounds (VOCs). It is a clean process allowing the extraction of proteins, sugars and other high quality co-products in the form of oil and/or aqueous extract, which can be used directly in cosmetics.

The grinding step requires adjusting the time and speed depending on the instrument used and the dry or wet nature of the material to be ground.

The water addition step requires adjusting the speed, time, water/solid ratio depending on the instrument to be used.

The enzymatic hydrolysis step requires determining the enzymes to be used and adjusting various parameters such as enzyme/substrate ratio, hydrolysis time, stirring speed, pH and temperature.

The endogenous or exogenous enzyme inactivation step requires the adjustment of parameters such as inactivation temperature, temperature, time, as well as pH.

The centrifugation step requires adjusting parameters such as speed, temperature, time and pH.

According to a particular embodiment, in step a), the appropriate particle size of the plant material is advantageously obtained by grinding said plant material. The grinding should be as fine as possible to promote the action of the enzymes. Ideally, all particles should have a size close to 50 μm, preferably close to 10 μm.

Preferably, the mass of water added to the plant material is 1 to 2 times the mass of said plant material and does not exceed this amount.

In particular in step b), the ratio of pectinase activity to cellulase activity being at least 0.14, preferably between 0.3 and 2.5, and more preferentially between 0.35 and 0.45, and the ratio of pectinase activity to hemicellulase activity being at least 7.10⁻³; preferably between 1.10⁻² and 0.5, and more preferentially between 1.10⁻² and 2.10⁻².

More particularly, the enzyme mixture used contains at least one cellulase, at least one hemicellulase, at least one pectinase, and advantageously also at least one protease. According to a particular embodiment, the cellulase(s), hemicellulase(s) and pectinase(s) represent 75% of the enzyme mixture and the protease(s) represent 25% of said enzyme mixture. According to a particular embodiment, the cellulases are present in a content ranging from 1% to 50%, preferably from 20% to 30% by weight of the enzyme mixture, the hemicellulases are present in a content ranging from 1% to 50%, preferably from 20% to 30% by weight of the enzyme mixture and the pectinases are present in a content ranging from 1% to 50%, preferably from 20% to 30% by weight of the enzyme mixture. And advantageously the proteases are present in a content ranging from 1% to 50%, preferably from 20% to 30% by weight of the enzyme mixture.

In a preferred embodiment the enzyme mixture used contains 25% cellulases, 25% hemicellulases, 25% pectinases and 25% proteases.

Preferably, the amount used of the enzyme mixture as defined above is between 0.25% and 10%, preferably between 1% and 6%, by volume of the water/plant material mixture.

Advantageously, the incubation according to step c) is carried out for 2 to 20 hours, preferably between 4 and 12 hours, at a temperature between 25° C. and 75° C., preferably between 40° C. and 60° C., and preferably around 50° C.

The hydrolysis reaction is then stopped by deactivating the enzymes by heating preferably between 80° C. and 105° C. for 5 to 20 minutes, for example.

Then the reaction medium is separated, according to step d), using all known suitable separation techniques, such as centrifugation or decanting.

According to a particular embodiment, the mixture is prefiltered on a 0.8 mm and then 0.5 mm sieve in order to remove the largest part of the solid phase, then a sterilizing filtration is advantageously carried out on clarifying plates with a pore size of 0.2 μm, at a pressure of 2 bar.

The aqueous phase obtained after sterilizing filtration is stabilized with preservatives (citric acid, potassium sorbate, sodium benzoate).

Thus, according to a preferred embodiment, said aqueous extract of rosewood, preferably of summer rosewood, is obtained by an extraction process comprising the steps:
a) addition of water to the pre-ground rosewood with a suitable particle size,
b) addition of an enzyme mixture containing at least one cellulase, at least one hemicellulase and at least one pectinase, and advantageously at least one protease,
c) incubation under stirring of the pre-ground rosewood and the enzyme mixture to release in the reaction medium oils if present, proteins and fermentable sugars, for a time depending on the desired yields,
d) separation of the reaction medium to obtain free oil, an aqueous phase containing proteins and fermentable sugars, and a solid phase, and
e) separation of the aqueous phase and optional addition of preservatives.

The aqueous extract of rosewood in accordance with the invention is characterized in particular by the presence of amino acids (aspartic acid, tyrosine, arginine) and total sugars (glucose, fructose).

The aqueous extract of rosewood in accordance with the invention is also characterized by the fact that it is not a fermented extract.

According to a particular embodiment, the aqueous extract of rosewood obtained in accordance with the invention comprises water in a content ranging from 94% to 96%, a dry extract content ranging from 1% to 6% and preferentially from 3% to 5% and preservatives in a content ranging from 0.5% to 1% by weight based on the total weight of the extract. The INCI name of this rosewood extract in accordance with the invention is water (and) rose extract (and) citric acid (and) potassium sorbate (and) sodium benzoate.

Composition and Galenic

The invention also relates to a cosmetic composition for topical application to the skin and/or lips, in particular the skin of the face and/or body, comprising, in a physiologically acceptable medium, at least an effective amount of at least one aqueous extract of rosewood in accordance with the invention.

‘Topical application’ is understood to mean a composition intended for application to keratinous materials. In fact, an oral composition is not intended for topical application to the skin.

The aqueous extract of rosewood is present in the cosmetic composition of the invention in an amount effective to obtain the desired effect.

In particular, it is present in a content ranging from 0.1% to 80%, in particular from 0.5% to 60% by weight of raw material based on the total weight of the composition. According to a first embodiment, it is present in a content ranging from 50% to 80%, in particular from 60% to 75% by weight of raw material based on the total weight of the composition. According to another particular embodiment, it is present in a content ranging from 0.1% to 20%, preferably from 0.5% to 10%, and still more preferably from 1% to 5% by weight of raw material based on the total weight of the composition. Illustrative examples are given below. The cosmetic composition of the invention may be in any galenic form suitable for topical application to keratinous materials and in particular the skin, for example a serum, an oil-in-water emulsion, a water-in-oil emulsion, a multiple emulsion, a microemulsion, a lotion, a suspension, an aqueous gel, a stick, a powder. The composition in accordance with the invention may be a care and/or make-up composition for keratinous materials.

“Keratinous material” is understood to mean the skin and/or its appendages, and the lips, in particular the skin of the face and/or body and the lips.

The composition is preferentially intended to be applied to the skin of the face and/or body or to the lips and is, for example, in the form of a lotion, a care cream, a serum, a facial fluid, a care gel for the face and/or body, a lip care composition, a lip balm, a lipstick, or a gloss.

According to a particular embodiment, the cosmetic composition in accordance with the invention is in the form of a care and/or make-up cream for the face.

According to another particular embodiment, the cosmetic composition in accordance with the invention is in the form of an aqueous gel for the eye contour.

According to another particular embodiment, the cosmetic composition in accordance with the invention is in the form of a care and/or make-up composition for the lips.

The aqueous phase generally represents from 1% to 99% by weight, based on the total weight of said composition.

Aqueous Phase

The aqueous phase of the composition in accordance with the invention comprises water and optionally a water-soluble solvent.

Water-soluble solvent' in accordance with the invention is understood to mean a compound which is liquid at room temperature and miscible with water (miscibility in water greater than 50% by weight at 25° C. and atmospheric pressure). Particular mention may be made of:
C₁-C₅ lower mono-alcohols such as ethanol, isopropanol and mixtures thereof;
C₂-C₈ glycols such as ethylene glycol, propylene glycol, 1,3-butylene glycol, dipropylene glycol, and mixtures thereof;
C₂-C₃₂ polyols such as polyglycerols, polyethylene glycols, and mixtures thereof,
   and mixtures thereof.

It may also include hydrophilic gelling agents, antioxidants, preservatives and mixtures thereof.

As hydrophilic gelling agents, particular mention may be made of acrylic acid polymers such as those with the INCI name Carbomer or the brand name Carbopol®, acrylic and methacrylic acid copolymers, carboxyvinyl polymers, associative thickeners of acrylic or polyurethane type, polysaccharide gelling agents such as alginates, natural or modified gums such as xanthan gums, carrageenan gums, agar gums, guar gums, gellan gums, chitosans, mannans, pullulans, cellulose derivatives, gelatin, pectins, inorganic gelling agents such as bentones or modified silicas, and mixtures thereof.

Natural or modified gum is understood to mean a polysaccharide, optionally modified, which hydrates in an aqueous medium to form a viscous solution or dispersion. Natural gums include algae extracts, plant exudates and gums extracted from seeds or plant roots and those obtained by microbiological fermentation. Modified or semi-synthetic gums include cellulose and starch derivatives and, in general, derivatives of all natural gums.

According to a particular embodiment, the cosmetic composition of the invention comprises a gelled aqueous phase, in particular by the presence of at least one acrylic polymer, in particular an acrylic acid polymer or copolymer of acrylic and methacrylic acid.

The content of hydrophilic gelling agent(s) in the cosmetic composition of the invention will generally range from 0.5% to 5%, in particular from 0.7% to 4%, preferably from 0.9% to 3% and even more preferably from 1% to 2% by weight based on the total weight of said composition.

Fat or Oil Phase

The cosmetic composition of the invention may further comprise a fat phase (solid fats) or an oil phase.

“Oil phase” is understood to mean an oil or a mixture of mutually miscible oils. “Oil” is understood to mean, in the sense of the invention, a fat which is insoluble in water and liquid at 25° C. and atmospheric pressure.

An oil phase in accordance with the invention may comprise hydrocarbon, silicone, fluorinated or non-fluorinated oils and mixtures thereof.

These oils can be volatile or non-volatile, vegetable, mineral or synthetic.

‘Hydrocarbon oil’ is understood to mean according to the invention an oil containing mainly hydrogen and carbon atoms.

‘Silicone oil’ is understood to mean according to the invention an oil comprising at least one silicon atom, and in particular at least one Si—O group.

‘Fluorinated oil’ is understood to mean according to the invention an oil comprising at least one fluorine atom.

As non-volatile hydrocarbon oils, particular mention may be made of hydrocarbon oils, hydrocarbon oils of vegetable origin, synthetic C₁₀-C₄₀ ethers, synthetic C₁₀-C₄₀ esters, C₁₂-C₂₆ fatty alcohols, C₁₂-C₂₂ higher fatty acids, and mixtures thereof.

As non-volatile silicone oils, particular mention may be made of phenylated silicone oils, non-phenylated silicone oils, and mixtures thereof.

The oils may be present in the composition of the invention in a content ranging from 0.01% to 95% by weight based on the total weight of the composition.

The fat or oil phase may also include lipophilic gelling agents, film-forming polymers, surfactants, antioxidants and mixtures thereof.

According to a particular embodiment of the invention, the cosmetic composition is in the form of a solution, an emulsion, a serum or a gel.

According to a particular embodiment, the cosmetic composition of the invention does not comprise any other rose extract than extracts of the Evanrat or Rose Jardin de Granville® variety.

Additional Ingredients

The composition of the invention can also comprise any additive usually used in cosmetics such as UV filters, antioxidants, fragrances, cosmetic active agents, such as for example emollient agents, moisturizing agents, vitamins, anti-aging agents, lifting agents, tightening agents, plumping agents, soothing agents, anti-pollution agents, lightening or depigmenting agents, fillers, pearlescent agents and mixtures thereof.

Thus, according to a particular embodiment, the invention relates to a cosmetic composition for topical application to the skin and/or lips, in particular the skin of the face and/or body, comprising, in a physiologically acceptable medium, at least an effective amount of at least one aqueous extract of rosewood in accordance with the invention and at least one cosmetic adjuvant selected from the group consisting of UV filters, antioxidants, fragrances, emollients agents, moisturizing agents, vitamins, anti-aging agents, lifting agents, tightening agents, plumping agents, soothing agents, anti-pollution agents, lightening or depigmenting agents, fillers, pearlescent agents and mixtures thereof.

According to a particular and preferred embodiment, the composition also comprises at least one cosmetic active agent selected from an antioxidant agent, a lifting agent, a tightening agent, a plumping agent, a soothing agent, an anti-pollution agent and/or a depigmenting agent. It can also include fillers and/or pearlescent agents, in particular white pearlescent agents, intended to mask color imperfections (dark circles) and illuminate the eyes.

Cosmetic Uses and Other Uses

The invention also relates to the non-therapeutic cosmetic use of at least one aqueous extract of rosewood in accordance with the invention, in particular of the Evanrat or Jardin de Granville® rose variety, as an agent intended to prevent and/or reduce the signs of skin aging, in particular the loss of tone, the loss of elasticity and/or the loss of firmness of the skin, the alteration of the barrier function, the appearance of wrinkles and/or fine lines, dry skin, and/or a dull and uneven complexion.

It also relates to a non-therapeutic cosmetic process intended to prevent and/or reduce the signs of skin aging, in particular the loss of tone, the loss of elasticity and/or the loss of firmness of the skin, the alteration of the barrier function, the appearance of wrinkles and/or fine lines, dry skin, and/or a dull and uneven complexion, characterized in that it comprises the application to the skin of the face and/or of the body and/or of the lips, of a cosmetic composition comprising an aqueous extract of rosewood as defined in the invention.

‘Skin and/or lips’ according to the invention is understood to mean in particular healthy skin and/or lips, i.e., not presenting conditions or disorders that would be related to a pathological state (‘non-healthy’ subjects, suffering from a pathology).

According to a particular embodiment, the composition is applied to dry, fragile, sensitive, stressed skin, and in particular tired skin having lost its firmness, its suppleness and its elasticity.

According to another particular embodiment, the composition is applied to dry, fragile, sensitive and/or stressed lips. According to a particular embodiment, the composition is applied to dry and/or fragile lips. According to another embodiment, to stressed lips. According to another embodiment, to sensitive lips.

Another subject matter of the invention is an aqueous extract of rosewood in accordance with the invention or a composition comprising same in accordance with the invention for use in soothing the skin, and/or preventing and/or treating skin disorders related to chronic microinflammation of the skin, in particular preventing and/or decreasing the signs of skin aging induced by chronic microinflammation of the skin.

According to this aspect of the invention, the aqueous extract of rosewood is used on skin with disorders related to chronic microinflammation, in particular inflammation due to endogenous and/or exogenous stresses having inflammaging effects.

The present invention further relates to an aqueous extract of rosewood in accordance with the invention or a composition comprising same for use in reducing solar lentigo.

According to this aspect of the invention, the aqueous extract of rosewood is used on skin with sun-related alterations.

The composition may be applied to the skin of the face and/or body or to the lips. According to a particular embodiment, the composition is applied to the face and/or body.

According to another aspect, the present invention relates to a rosewood extract in accordance with the invention, or a composition containing same, for soothing the skin, and/or preventing and/or treating skin disorders related to chronic microinflammation of the skin.

The present invention further relates to a rosewood extract in accordance with the invention, or a composition containing same for reducing solar lentigo.

According to another aspect, the present invention relates to a method for treating keratinous materials, preferably the skin and/or lips, to soothe the skin, and/or to prevent and/or treat skin disorders related to chronic microinflammation of the skin, comprising the administration to a person in need thereof of a rosewood extract in accordance with the invention, or a composition containing same.

The invention also relates to a method for treating keratinous materials, preferably the skin and/or lips, to reduce solar lentigo, comprising the administration to a person in need thereof of a rosewood extract in accordance with the invention, or a composition containing same.

The invention will now be illustrated in the following non-limiting examples. Unless otherwise indicated, % are expressed as % by weight based on the total weight of the composition.

### EXAMPLES

### Example 1

### Extraction from Rosewood and Analysis of These Extracts

Preparation of the plant

Rosewood and rose flowers of the Evanrat variety, in particular the Jardin de Granville® rose variety, are used.

The raw materials, in this case rosewood, optionally with added rose flowers, were reduced to powder in three steps.
Step 1: passage through a garden shredder to obtain two-centimeter sections
Step 2: passage through an industrial grinder

Putting into Solution

The enzymatic extraction was performed in a reactor with a maximum capacity of 70 L. The reactor is stirred by a propeller and is thermostatically controlled by a double jacket.

16 kg of rosewood powder with 0.1% rose flowers is dispersed and suspended in 47.5 kg of water.

In order to deactivate the endogenous enzymes, the mixture was raised to a temperature of 85° C. for 1 min and then stabilized at 50° C.

Lysis of Plant Walls

0.8 kg of a HEL1PR1 enzyme cocktail, comprising a mixture of 75% cellulases, hemicellulases and pectinases and 25% proteases, was added to the corresponding reaction mixture (5% w/w). The reaction was continued for 4 hours, the temperature was maintained at 50° C.

Enzyme Inactivation

The mixture was heated to 85° C. for 1 min to inactivate the enzymes and then returned to 35° C.

Pre-Filtration

The mixture is prefiltered on a 0.8 mm and then 0.5 mm sieve to remove the largest part of the solid phase, then on a sheet.

Sterilizing Filtration

A filtration was carried out on clarifying plates (PALL Corporation) with a pore size of 0.2 μm, at a pressure of 2 bar.

Formulation

The aqueous phase obtained after sterilizing filtration is stabilized with the following preservatives:
Citric acid
Potassium sorbate and
Sodium benzoate

After filtration, an aqueous extract of rosewood with 3.7% by weight of dry matter based on the total weight of the extract is obtained. This extract is used in the following examples to demonstrate its effects on the skin; the following examples use cultures of keratinocytes and/or melanocytes.

Quantification assays performed on this aqueous extract not stabilized by preservatives give the following results:

Total polyphenols (g/L): 2.5

Reducing sugars (g/L): 16.2

Protein nitrogen (g/L): 0.64

Total protein (g/L): <6

Glucose (g/100 g): 1.4 (±0.8)

Fructose (g/100 g): 0.3 (±0.3)

### Example 2

### Protocol for Studying the Expression of Genes of Interest in Keratinocyte Culture

Normal human keratinocytes (NHK) are cultured and then treated with the aqueous extract of rosewood as prepared in Example 1.

After processing the NHK, a TaqMan low-density array (TLDA) study on the studied genes is performed from the cDNAs obtained after reverse transcription of the extracted total RNAs.

The aqueous extract of rosewood is tested at 0.1% or 0.25% by weight of raw material (excipient water).

NHK Culture

Normal human keratinocytes are derived from a skin sample obtained from plastic surgery. The cells are cultured in complete Epilife medium at a seeding density of 50,000 cells per well in 12-well plates. At subconfluence, the cells are treated for 24 hours with the doses of rose extracts described above.

TaqMan Low-Density Array (TLDA) Technology

Obtaining Total RNA

The cell culture medium is removed, and 250 μL of RLT lysis buffer (provided in the Nucleospin RNA Trace kit, Macherey-Nagel) is added. The cells are scraped with a Cell Scraper and the cell lysate is collected in a 1.2 mL deepwell plate (provided in the Nucleospin RNA kit). Total RNA is extracted according to the defined protocols.

The total RNA solutions obtained are assayed, and their quality checked, using a microplate reader, the spectrostar NANO (BMG Labtech) coupled to the Microlab STAR. This device is connected to the computer controlling the automated liquid handler and has the specific software for the analysis of the results (MARS software). The technique requires a 384-well microplate (LoBase), a positive control (RNA 250, AM7155, Thermofisher) to validate the pipetting performed by the automated liquid handler and the values generated by the spectrostar NANO reader.

Synthesis of Complementary DNA

The reverse transcription (RT) kit used was the High-Capacity Reverse Transcription Kit (Thermo Fisher). It was used according to the protocol provided. 500 ng of total RNA is diluted in water to a final volume of 25 μL and then incubated for 10 minutes at 25° C. and 2 hours at 37° C. in the presence of 25 μL of High-Capacity Reverse Transcription Kit 2× reaction mix previously prepared as described below. The different incubations are done in the TRobot (Biometra).

TABLE 1
High-Capacity Reverse Transcription Kit 2X reaction mix for 1 reaction

| Reagents | RT buffer | dNTP | Primer | RNase OUT | RT | H2O |
|---|---|---|---|---|---|---|
| Volume | 5 μL | 2 μL | 5 μL | 0.5 μL | 2.5 μL | 10 μL |

PCR-TaqMan Low-Density Array

15 μL of each RT is mixed with 60 μL of water and 75 μL of TaqMan Gene Expression master mix (ThermoFisher), containing the DNA polymerase, is added. After homogenization, 100 μL is deposited on the microfluidic cards containing the probes corresponding to the tested genes (Table 2 below), which are centrifuged and sealed. The CD Rom corresponding to the profile of the genes deposited on the plates is loaded into the SDS 2.3 software, indicating the location of each gene on the card. The control gene (or “endogenous”) to be used for the normalization of the results is indicated before the PCR is launched. The latter is performed according to the protocol provided by Applied Biosystems in the ABI Prism 7900HT Sequence Detection System. The qPCR steps are 2 min at 50° C., 10 min at 94.5° C. then 30 s at 97° C. and 1 min at 59.7° C. for 40 cycles. Depending on the effects sought, the person skilled in the art determines which genes are relevant to be included in the study; it is up to his or her general knowledge to determine and optimize for each gene the nucleotide sequences of the PCR primers to be used.

Statistical Analysis

Real-time quantitative PCR can be used if its efficiency is between 90% and 110%. For each sample, the number of cycles at which the signal appears is determined by the SDS 2.3 software. For the same assay, the expression levels of the transcripts of interest obtained are normalized to the value obtained for the beta-2-microglobulin housekeeping gene. This gene, whose expression is constitutive and invariant, makes it possible to avoid all variations induced during the experiment (total RNA assay, pipetting, reverse transcription step, PCR in the apparatus).

In the TLDA RT-PCR method, quantification is performed using the comparative ΔΔCt method. The relative quantification (RQ) values obtained correspond to the level of amplitude (x times more or less than the control) of the expression compared with our control, here the unirradiated. The RQ is obtained by the following calculation where the control is equal to 1:

RQ=2^{-ΔΔCt}=2^{-(ΔCt treated-ΔCt untreated)}

ΔCt treated=Ct target gene treated−Ct housekeeping gene treated

ΔCt untreated Ct target gene untreated−Ct housekeeping gene untreated

In order to evaluate statistically significant variations in transcriptional activity, we will use Student's t-test. Each condition is performed in triplicate (3 untreated and 3 treated under the same conditions). Fischer's F-test is first applied by comparing the two data matrices. When the value is greater than α=0.05 then the variance for Student's t-test is 2, when the Fischer F-test is less than α=0.05 then the variance will be equal to 3. The transcriptional variations retained will be those with a Student's t-test less than α=0.05.

Results are presented as the mean with n=3. Student's t-test was used to compare the effect between treated and untreated cells.

Results are considered significant for p<0.05(*) or p<0.01(**).

The results are presented in the following Table 3:

### Example 3

### Effect of Aqueous Extract of Rosewood on Oxidative Stress

We studied the effect of rosewood extract in accordance with the invention on the stimulation of gene expression of endogenous antioxidant enzymes, which participate in neutralizing free radicals and thus protect the skin cells in particular against aging. This study was carried out on keratinocyte culture following the protocol detailed in Example 2.

TABLE 2
Item codes for the genes under study

| | | TaqMan | RefSeq GenBank |
|---|---|---|---|
| Gene | Symbol | item code | accession number |
| Methionine sulfoxide | SELX | Hs00249482_m1 | NM_016332.2 |
| reductase B1 | | | |
| Glutathione peroxidase 4 | GPX4 | Hs00157812_m1 | NM_001039847.2 |
| Catalase | CAT | Hs00156308_m1 | NM_001752.3 |

TABLE 3
Modulation of the expression of genes of interest after
treatment with rosewood extract in accordance with the invention

| | | 0.1% aqueous |
|---|---|---|
| Gene | Symbol | extract of rosewood |
| Methionine sulfoxide reductase B1 | SELX | +63% |
| Glutathione peroxidase 4 | GPX4 | +52% |
| Catalase | CAT | +29% |

These results show that the aqueous extract of rosewood in accordance with the invention stimulates genes involved in the response to oxidative stress, and thus limits the accumulation of free radicals. The aqueous extract of rosewood is therefore advantageous for use in preventing and/or reducing skin reactions caused in particular by the environment, pollution and tobacco, and thus in combating cellular aging of the skin.

### Example 4

### Effect of Aqueous Extract of Rosewood on the Barrier Function

We studied the effect of rosewood extract in accordance with the invention on the expression of genes involved in cell cohesion, epidermal differentiation, or epidermal homeostasis and thus participating in the regulation of the skin barrier function. This study was carried out on keratinocyte culture following the protocol detailed in Example 2.

TABLE 4
Item codes for the genes under study

| | | | RefSeq GenBank |
|---|---|---|---|
| Gene | Symbol | TaqMan item code | accession number |
| Transglutaminase | TGM1 | Hs00165929_m1 | NM_000359 |
| Kallikrein related | KLK7 | Hs00192503_m1 | NM_001207053.1 |
| peptidase 7 | | | NM_001243126.1 |
| | | | NM _005046.3 |
| | | | NM_139277.2 |
| Cornifin | SPRR b | Hs00824893_m1 | NM_003125.2 |
| Ceramide synthase 3 | CERS3 | Hs00698859_m1 | NM_001290341.2 |
| | | | NM_001290342.2 |
| | | | NM_001290343.1 |
| | | | NM_178842.4 |
| Cytokeratin 1 | KRT1 | Hs00196158_m1 | NM_006121.3 |
| Connexin 43 | GJA1 | Hs00748445_s1 | NM_000165.4 |
| Desmoglein 1 | DSG1 | Hs00170047_m1 | NM_001942.3 |
| Occludin | OCLN | Hs00170162_m1 | NM_001205254.2 |
| Claudin | CLDN1 | Hs00221623_m1 | NM_021101.4 |
| Fatty Acid Binding | FABP5 | Hs00192700_m1 | NM_005094.3 |
| Protein 5 | | | |
| Aquaporin 9 | AQP9 | Hs00175573_m1 | NM _002638.3 |

TABLE 5
Modulation of the expression of genes of interest after treat-
ment with rosewood extract in accordance with the invention

| | | 0.1% aqueous | |
|---|---|---|---|
| | | extract of | 0.25% aqueous |
| Gene | Symbol | rosewood | extract of rosewood |
| Transglutaminase | TGM1 | +304% | +460% |
| Kallikrein related | KLK7 | +172% | +174% |
| peptidase 7 | | | |
| Cornifin | SPRR b | +334% | +436% |
| Ceramide synthase 3 | CERS3 | +156% | +111% |
| Cytokeratin 1 | KRT1 | +905% | +866% |
| Connexin 43 | GJA1 | +132% | +117% |
| Desmoglein 1 | DSG1 | +545% | +493% |
| Occludin | OCLN | +72% | +87% |
| Claudin-1 | CLDN1 | +178% | +152% |
| Fatty Acid Binding | FABP5 | +271% | +309% |
| Protein 5 | | | |
| Aquaporin 9 | AQP9 | +858% | +1364% |

These results show that the aqueous extract of rosewood in accordance with the invention stimulates many mediators of epidermal differentiation and cell cohesion. The aqueous extract of rosewood in accordance with the invention is therefore advantageous for promoting and/or stimulating the skin barrier function and in particular for promoting skin firmness, suppleness and elasticity.

### Example 5

### Effect of the Aqueous Extract of Rosewood on Melanin Transfer

The objective of this study is to investigate the estimated depigmenting effect of the rosewood extract in accordance with the invention. This evaluation is carried out by measuring the melanin transfer rate in a keratinocyte/melanocyte co-culture model.

NHK/NHM Co-Culture Model

NHK are seeded at 50,000 cells/well in 1 mL of a mixture of Epilife and M254 medium (Invitrogen), respectively 25% and 75%, in 24-well culture media.

After 24 hours of culture, NHM are added at 4000 cells/well in 400 μL/well in the same renewed medium.

Cell Treatment

After 24 hours of culture at 37° C., the co-culture is treated or not with active agents (rosewood extract in accordance with the invention or a positive control).

An untreated control and a positive control, niacinamide (Sigma) at a concentration of 10 μmol/L are included in the study.

The cells are treated for 48 hours without renewal with 0.1% rosewood extract in accordance with the invention or with niacinamide for the positive control condition.

Immunostaining

After 48 hours of treatment, the culture medium is removed. The cells are rinsed with PBS (Invitrogen), then fixed with formalin (Sigma) for 10 minutes.

After rinsing with PBS, the culture wells are filled with 0.1% PBS/Triton solution (Sigma) for 10 minutes to permeabilize the fixed cell membranes, then rinsed twice with PBS.

The cells are covered with a 1% PBS/BSA solution (Sigma). After 30 minutes at room temperature, the PBS/BSA solution is removed. The cells are then covered with a solution of primary anti-melanoma marker antibody HMB45 (Dako, Mouse) diluted 1:150 in 1% PBS-BSA solution, for 2 hours at room temperature. The cells are then rinsed 3 times in PBS solution.

The cells are then covered with a secondary anti-mouse antibody solution (Alexa fluor 568, goat anti-mouse, Invitrogen) diluted 1:300 in a 1% PBS-BSA solution. Cell nuclei are stained with DAPI diluted 1:100 (Invitrogen) and actin is stained with Alexa Fluor 488 phalloidin diluted 1:100 in the secondary antibody solution. The cells are incubated for 60 minutes at room temperature protected from light. The culture wells are rinsed 3 times in PBS and then a few drops of mounting medium (DAKO) are deposited on the cells.

Plates are kept at 4° C. and in the dark until image acquisition.

Image Acquisition by HCS

Plates are scanned on the Thermo Cellomics ArrayScan XTi according to the supplier's recommendations.

Image Analysis

Images are analyzed using the Cell Profiler analysis software. A program is created to quantify the number of melanosomes transferred per keratinocyte surface area. The results are transferred directly into a Microsoft Excel file.

Statistical Analysis

The statistical analysis of the results is done using Microsoft Excel software.

This analysis consists of a calculation of the mean, standard deviation and confidence interval (α=0.05) of the number of melanosomes transferred per keratinocyte surface area. In order to evaluate statistically significant variations, Student's T-test is used. Fischer's F-test is first applied by comparing the two data matrices. When the value is greater than α=0.05 then the variance for the Student's t-test is 2, when the Fischer's F-test is less than α=0.05 then the variance will be equal to 3. The variations retained will be those with a Student's t-test less than α=0.05.

Results

The positive control as well as the different doses of tested ingredients are compared with the untreated control. A decrease in melanosome transfer of −30% is observed for the positive control (niacinamide) compared with the untreated control. Concerning rosewood extract at a dose of 0.1%, a significant decrease of −30% in the number of melanosomes transferred per keratinocyte was observed compared with the untreated control, i.e., a level equivalent to the niacinamide positive control.

This study therefore highlights the depigmenting effect of the rosewood extract. The use of this extract is therefore particularly advantageous for preventing and/or treating the uneven appearance of the complexion, the appearance of cutaneous pigment spots, or age spots, in particular on the back of the hands, on the face, the neckline or, in men, the skull.

### Example 6

### Effect of the Aqueous Extract of Rosewood on Innate Immunity

We studied the effect of the rosewood extract in accordance with the invention on the expression of genes encoding antibacterial peptides and therefore involved in innate immunity. This study was carried out on keratinocyte culture following the protocol detailed in Example 2.

TABLE 6
Item codes for the genes under study

| | | | RefSeq |
|---|---|---|---|
| | | | GenBank accession |
| Gene | Symbol | TaqMan item code | number |
| Beta Defensin 4 | DEFB4 | Hs00175474_m1 | NM_004942.3 |
| Beta Defensin 3 | DEFB3 | Hs00218678_m1 | NM_018661.4 |
| Elafin | PI3 | Hs00160066_m1 | NM_002638.3 |

TABLE 7
Modulation of the expression of genes of interest after treat-
ment with the rosewood extract in accordance with the invention

| | | 0.1% aqueous | 0.25% aqueous |
|---|---|---|---|
| Gene | Symbol | extract of rosewood | extract of rosewood |
| Beta Defensin 4 | DEFB4 | >10,000% | >10,000% |
| Beta Defensin 3 | DEFB3 | — | +74.5% |
| Elafin | PI3 | +78% | +484% |

In this study, a strong stimulation of the expression of genes encoding antibacterial peptides was observed, which highlights the beneficial effect of the rosewood extract in accordance with the invention on its ability to promote the innate immunity of the skin.

### Example 7

### Effect of the Aqueous Extract of Rosewood on Microinflammation

We studied the effect of rosewood extract in accordance with the invention on the expression of cytokines or cytokine receptors, which are genes involved in microinflammation processes in the skin that can promote skin aging or inflammaging phenomena. This study was carried out on keratinocyte culture following the protocol detailed in Example 2.

TABLE 8
Item codes for the genes under study

| | | | RefSeq |
|---|---|---|---|
| | | | Accession number |
| Gene | Symbol | TaqMan item code | GenBank |
| Interleukin 1 alpha | IL-1A | Hs00174092_m1 | NM_000575.4 |
| Interleukin 1 beta | IL-1B | Hs00174097_m1 | |
| Tumor necrosis factor | TNF | Hs00174128_m1 | NM_000594.3 |
| Interleukin 1 receptor | IL-1R2 | Hs01030389_m1 | NM_004633.3 |
| type II | | | |
| Interleukin 4 receptor | IL-4R | Hs00166237_m1 | NM_000418.3 |
| Interleukin 1 Receptor | IL-1RAP | Hs00370511_g1 | NM_001167928.1 |
| Accessory Protein | | | NM_001167929.1 |
| | | | NM_001167930.1 |
| | | | NM_001167931.1 |
| | | | NM_002182.3 |
| | | | NM_134470.3 |
| Prostaglandin | PTGER4 | Hs00168761_m1 | NM_000958.2 |
| receptor 4 | | | |
| Vascular endothelial | VEGFA | Hs03929005ml | NM_001025370.2 |
| growth factor A | | | NM_001171628.1 |
| Suppressor of | SOCS1 | Hs00864158_g1 | NM_003745.1 |
| cytokine signaling | | | |
| protein 1 | | | |

TABLE 9
Modulation of the expression of genes of interest after treat-
ment with the rosewood extract in accordance with the invention

| | | 0.1% aqueous |
|---|---|---|
| | | extract |
| Gene | Symbol | of rosewood |
| Interleukin 1 alpha | IL-1A | −69.5% |
| Interleukin 1 beta | IL-1B | −68% |
| Tumor necrosis factor | TNF | −41% |
| Interleukin 1 receptor type 11 | IL-1R2 | −30% |
| Interleukin 4 receptor | IL-4R | −34% |
| Interleukin 1 Receptor Accessory Protein | IL-1RAP | −38% |
| Prostaglandin receptor 4 | PTGER4 | −50.5% |
| Vascular endothelial growth factor A | VEGFA | −47% |
| Suppressor of cytokine signaling protein 1 | SOCS1 | +104% |

These results show that the aqueous extract of rosewood in accordance with the invention inhibits numerous mediators of inflammation, in particular cytokines and cytokine receptors.

The aqueous extract of rosewood in accordance with the invention is therefore advantageous for use in preventing and/or reducing the effects of chronic microinflammation of the skin and thus combating skin aging or inflammaging.

Conclusion

The whole of the effects observed on oxidative stress, barrier function, pigmentation, immunity and microinflammation show that the rosewood extract in accordance with the invention is of interest to prevent and/or treat the main factors responsible for skin aging and thus to improve the appearance of aged skin.

### Example 8

### Formulations

8.1 Composition in the Form of an Emulsion

| Aqueous phase: | |
|---|---|
| Demineralized water | qs 100.0% |
| Glycols | 20.0% |
| Preservatives | 0.6% |
| Chelating agent | 0.04% |
| Carbomer (Carbopol ® 981) | 0.3% |
| Sodium polyacrylate (Covacryl ® MV60) | 0.2% |
| Sodium Hydroxide | 0.15% |
| Aqueous extract of rosewood according to the invention* | 3.0% |
| Fat phase: | |
| Vegetable oil, esters, silicones | 16% |
| Antioxidant | 0.2% |
| Fragrance concentrate | 0.4% |
| Steareth-2 | 0.8% |
| Steareth-21 | 1.5% |

*as described in Example 1

The composition is prepared according to the following procedure:
the gelling agents are dispersed in the aqueous phase (excluding aqueous rosewood extract and sodium hydroxide) which is heated to 70° C.;
the fat phase (excluding fragrance concentrate and antioxidant) is heated to 70° C.;
the emulsion is made by introducing the fat phase into the aqueous phase under strong stirring;
the gelling agents are neutralized by adding sodium hydroxide and the emulsion is cooled under moderate stirring with the introduction of the fragrance concentrate, the antioxidant and the aqueous extract of rosewood at low temperature.

Applied to the skin, particularly the face, this composition gives tone, elasticity and firmness, the features are enhanced, the face is reshaped.

8.2 Composition in the Form of a Solid Dispersion of Fats, in Spherical or Spheroid Form

| Aqueous phase | |
|---|---|
| Demineralized water | qs 100% |
| Aqueous extract of rosewood according to the invention* | 3.00% |
| Phenoxyethanol | 0.42% |
| Xanthan gum | 0.36% |
| Fat phase | |
| C10-18 triglycerides | 38.84% |
| Antioxidant | 0.1% |

*as described in Example 1

The composition is prepared according to the following procedure:
the wax (C10-18 triglycerides=Lipocire from Gatefossé) is heated above its melting point (a few degrees) with the antioxidant;
the melted fat phase is poured under stirring into water previously heated to the same temperature as the fat phase;

the whole is kept under stirring by a rotary system for a few minutes until the desired droplet size is obtained;
the dispersion obtained is rapidly cooled by adding pre-cooled glycol water (about −4° C.) to solidify the lipid spheroids;
the stirring is stopped when the spheroids are solidified and then recovered at the surface or filtered;
the aqueous phase is prepared by mixing water, xanthan gum, the preservative and the aqueous extract of rosewood in accordance with the invention;
the spheroids are recovered from the surface and incorporated into the xanthan gel containing the aqueous extract of rosewood in accordance with the invention.

Applied to the skin, this composition helps to reduce the visible signs of aging, in particular by giving tone and firmness to the skin; the oval of the face is as if redesigned.

8.3: Composition in the Form of an Aqueous Gel for the Face

| | |
|---|---|
| Aqueous extract of rosewood according to the | 80.00% |
| invention* | |
| Preservatives | 0.50% |
| Sugar | 0.20% |
| Carbomer | 0.70% |
| Purified water | qs 100% |
| Tetrasodium EDTA powder | 0.20% |
| Sodium hydroxide | 0.17% |

*as described in Example 1

The rosewood extract in accordance with the invention and the preservatives are mixed and homogenized at room temperature under stirring. The sugars are added under stirring, then the carbomer, then water and EDTA under stirring. The aqueous gel is then neutralized with the addition of sodium hydroxide under stirring until a homogeneous gel is obtained.

Applied to the skin of the face, this aqueous gel corrects skin dryness, and dull or uneven complexion, and contributes to the youthfulness of the skin.

8.4: Composition in the Form of a Micronutrient Gel-Serum for the Eye Contour

| Purified water | qs 100.00% |
|---|---|
| Glycols | 13.0% |
| Preservatives | 0.60% |
| Carbomer | 0.80% |
| Glyceryl stearate citrate | 0.70% |
| Lecithin and sodium acrylates copolymer | 1.20% |
| (Lecigel PCR negative) | |
| lsostearate isostearyl | 9.0% |
| Bis-diglyceryl polyacyladipate-2 | 1.0% |
| (Softisan 649 MB) | |
| Silica | 2.0% |
| Pearlescent agents | 1.0% |
| Centella asiatica extract | 0.5% |
| Horse chestnut extract | 0.1% |
| Aqueous extract of rosewood in accordance | 3.0% |
| with the invention* | |
| Tocopheryl acetate | 0.10% |
| Rose fragrance | 0.20% |

*as described in Example 1

The ingredients of the aqueous phase (water, glycols and carbomer) are mixed at 80° C. under stirring. The preservatives are then added, followed by the gelling agents at 80° C. under stirring, then the temperature is lowered to 75° C. Then the surfactants are emulsified in the aqueous phase under stirring. The fillers and pearlescent agents, previously pasted and homogenized, are added at 40° C. The cryoextract, the aqueous extract of rosewood in accordance with the invention, is then added at 40° C. under stirring until 30° C.

After application to the eye contour area, this gel-serum helps smooth out wrinkles and fine lines, the eye area is firmed up, the appearance of dark circles and puffiness is reduced. The youthful appearance of the eyes is restored with each application.

8.5 Lip Balm

| | |
|---|---|
| Silica | 10% |
| Nylon powder | 7% |
| Polyethylene wax | 17% |
| Glycerin | 14% |
| Aqueous extract of rosewood in accordance | 3.0% |
| with the invention* | |
| Isopropyl myristate | qs 100% |

This composition is prepared by mixing the ingredients according to conventional balm formulation methods.

This lip balm, after application on dry and/or fragile lips, moisturizes and smooths them.

1. A cosmetic composition for topical application to the skin and/or lips, in particular the skin of the face and/or body, comprising, in a physiologically acceptable medium, at least an effective amount of at least one aqueous extract of rosewood and, in particular, at least one cosmetic adjuvant selected from the group consisting of UV filters, antioxidants, fragrances, emollient agents, moisturizing agents, vitamins, anti-aging agents, lifting agents, tightening agents, plumping agents, soothing agents, anti-pollution agents, lightening or depigmenting agents, fillers, pearlescent agents and mixtures thereof.

2. The cosmetic composition as claimed in claim 1, characterized in that the aqueous extract of rosewood is present in the composition in a content ranging from 0.1% to 80%, in particular from 0.1% to 20%, preferably from 0.5% to 10% and still more preferably from 1% to 5% by weight of raw material based on the total weight of said composition.

3. The cosmetic composition as claimed in claim 1 or claim 2, characterized in that it further comprises at least one cosmetic active agent selected from an antioxidant agent, a lifting agent, a tightening agent, a plumping agent, a soothing agent, an anti-pollution agent and/or a depigmenting agent.

4. A non-therapeutic cosmetic process intended to prevent and/or reduce the signs of skin aging, in particular the loss of tone, the loss of elasticity and/or the loss of firmness of the skin, the alteration of the barrier function, the appearance of wrinkles and/or fine lines, dry skin and/or a dull and uneven complexion, characterized in that it comprises the application to healthy skin, in particular to the skin of the face and/or body, of a cosmetic composition comprising an aqueous extract of rosewood as defined in any one of claims 1 to 3.

5. An aqueous extract of rosewood, preferably of summer rosewood, obtained by enzymatic extraction using a cosmetically acceptable polar solvent, characterized in that the roses are of the Evanrat variety, in particular of the Jardin de Granville® rose variety.

6. The extract as claimed in claim 5, characterized in that it is obtained by an enzymatic extraction process comprising the steps:
a) addition of water to the pre-ground rosewood with a suitable particle size,
b) addition of an enzyme mixture containing at least one cellulase, at least one hemicellulase and at least one pectinase, and advantageously also at least one protease,
c) incubation under stirring of the pre-ground rosewood and the enzyme mixture to release in the reaction medium oils, proteins and fermentable sugars, for a time depending on the desired yields,
d) separation of the reaction medium to obtain free oil, an aqueous phase containing proteins and fermentable sugars, and a solid phase,
e) separation of the aqueous phase and optional addition of preservatives.

7. A process for preparing a rosewood extract, preferably of summer rosewood, as defined in claim 5 or claim 6 comprising the steps:
a) addition of water to the pre-ground rosewood with a suitable particle size,
b) addition of an enzyme mixture containing at least one cellulase, at least one hemicellulase and at least one pectinase, and advantageously also at least one protease,
c) incubation under stirring of the pre-ground rosewood and the enzyme mixture to release in the reaction medium oils, proteins and fermentable sugars, for a time depending on the desired yields,
d) separation of the reaction medium to obtain free oil, an aqueous phase containing proteins and fermentable sugars, and a solid phase,
e) separation of the aqueous phase and optional addition of preservatives.

8. Non-therapeutic cosmetic use of at least one aqueous extract of rosewood as defined in claim 5 or claim 6, as an agent intended to prevent and/or reduce the signs of skin aging, in particular the loss of tone, the loss of elasticity and/or the loss of firmness of the skin, the alteration of the barrier function, the appearance of wrinkles and/or fine lines, dry skin, and/or a dull and uneven complexion.

9. The aqueous extract of rosewood as defined in claim 5 or claim 6 or a composition comprising same as defined in any one of claims 1 to 3 for use in soothing the skin, and/or preventing and/or treating skin disorders related to chronic microinflammation of the skin.

10. The aqueous extract of rosewood as defined in claim 5 or claim 6 or a composition comprising same as defined in any one of claims 1 to 3 for use in reducing solar lentigo.
